(19) United States
(12) Patent Application Publication
(10) Pub. No.: US 2026/0324953 A1
(43) Pub. Date: Oct. 22, 2026

(54) Drying Swab for Endoscope Channels

(71) Applicant: Cygnus Medical, LLC, Branford, CT (US)

(72) Inventor: Shaun Sweeney, Wayne, NJ (US)

(21) Appl. No.: 19/183,046

(22) Filed: Apr. 18, 2025

(51) Int. Cl. A61B 1/00

(52) U.S. Cl. CPC A61B 1/00131

(57) ABSTRACT

A drying device for a channel or lumen of an endoscope having a flexible leader having a first end and a second end. A first swab formed of a hydrophobic non-absorbent foam, and a second swab formed of a hydrophilic foam are affixed to the flexible leader so they can be passed sequentially through the channel or lumen. A method of drying a channel or lumen of an endoscope includes passing the drying device leader through the channel or lumen; and pulling on the leader to cause the first swab to wipe the walls of the channel or lumen and push liquid out of the channel or lumen; and further pulling on the leader to cause the second swab to wipe the walls of the channel or lumen and absorb liquid in the channel or lumen.

### FIELD OF THE INVENTION

The invention relates to the field of medical devices, and in particular, the cleaning and sterilization of reusable surgical instruments such as endoscopes. Specifically, the invention relates to a flexible cleaning device for use to dry endoscope lumens or channels after cleaning and sterilization.

### BACKGROUND OF THE INVENTION

Each surgical operation leaves biological residue from the patient's body on the surgical instruments. Medical professionals at hospitals and other surgical facilities have long been aware of the risk that small fragments of tissue from a prior case may remain on a grasping platform or cutting blades and have developed protocols to remove biological residue and then to sterilize the surgical instruments.

Reusable surgical instruments, such as flexible and nonflexible endoscopes, must be cleaned and sterilized after every procedure, before they are reused. Endoscopes are typically processed according to procedures for high-level disinfection (“HLD”) which has the goal of disinfection of all microorganisms in or on an instrument.

Typically cleaning and sterilization are two separate steps, with cleaning being performed to remove body tissues and fluids, followed by sterilization to sanitize the instruments and destroy any potential pathogens that were acquired during use of the instrument. In a HLD sterilization step, the instrument is immersed in a high-level disinfectant solution such as an ortho-phthalaldehyde disinfectant or a hydrogen peroxide disinfectant for a recommended time period. The instrument is then rinsed. After rinsing, the channels in the instrument are dried.

A common issue that arises after cleaning and High Level Disinfection or sterilization of endoscopes is the presence of residual liquid trapped within the lumens or channels of the endoscope. Residual water located within the instrument is undesirable as it can encourage pathogen growth. Wet surfaces may support bacterial or fungal development in the instrument, potentially exposing a patient to such pathogens. However, it is very difficult to remove all residual water from the long narrow channels of a typical endoscope.

Channels in endoscopes typically have a diameter in the range of about 2.0 mm to about 4.5 mm, often typically 3.0 mm. The current approaches are not effective because the endoscope channels or lumens can be 2 meters long but have a 3.0 mm diameter. The surface tension of water is very high, so that the water remains trapped in the instruments despite efforts at drying.

Flexible endoscopes have been found to harbor potential pathogens such as Pseudomonas aeruginosa, Escherichia coli, Citrobacter freundii, Stenotrophomonas maltophilia, Mycobacterium avium, and mold, even after high-level disinfection (HLD). Pathogens which survive HLD can propagate in a wet environment, potentially leading to a biofilm plaque that makes cleaning even more difficult, as such biofilm resists removal by brushing, disinfection, or drying.

In the past, air drying while hanging the endoscope was the established procedure to dry instruments, but studies have shown that air drying is insufficient to completely dry the channels inside an instrument.

Use of alcohol or other solvents to absorb moisture has not been shown to be effective either. Many automated endoscope preprocessors (AERs) have alcohol flush and air purge cycles to aid in drying. These drying methods have been evaluated but have been determined to not be effective. One study evaluated automated endoscope reprocessor (AER) alcohol flush and air purge cycles and found that 100% (42/42) of endoscopes were still wet after such a drying cycle.

Consequently, standards and guidelines now direct medical professionals to purge and dry instrument channels for at least 10 minutes with pressure-regulated, forced “instrument air” or high-efficiency particulate (HEPA)-filtered air. However, even this protocol is not always effective, particularly if there is simethicone or alcohol present in the instrument channels.

Medical research groups have continued to experiment with optimization of endoscope drying through implementations of various air flow regimes, however, currently, there is no commercially available product or method that provides a consistent drying of instrument channels in medical instruments such as endoscopes.

Accordingly, it would be desirable to provide a device which is effective in drying the lumens and channels of endoscopic instruments after cleaning and sterilization.

Furthermore, it would be desirable to provide a device which is effective to dry larger biopsy channels in an instrument such as an endoscope, freeing up a greater volume of air for use to dry smaller channels in the endoscope.

### SUMMARY OF THE INVENTION

It is an object of the present invention to provide a drying device for a channel or lumen of an endoscope which is effective in providing a consistent and thorough drying of a channel or lumen of an endoscope.

It is an object of the present invention to provide a method of drying a channel or lumen of an endoscope.

A drying device for a channel or lumen of an endoscope in accordance with the invention has a flexible leader having a first end and a second end. A first swab formed of a hydrophobic non-absorbent foam is affixed to the flexible leader at a position whereby the distance from the flexible leader first end to the first swab is greater than the length of the channel or lumen. The first swab position is located away from the flexible leader second end. A second swab formed of a hydrophilic foam is affixed to the flexible leader between the first swab and the second end.

A method of use of the drying device includes steps of passing the first end of the leader through the first open end of the channel or lumen until the first end of the leader passes out of the channel or lumen from the second end of the channel or lumen; pulling on the first end of the leader to cause the first swab to wipe the walls of the channel or lumen and push liquid in the channel or lumen out of the second end of the channel or lumen; and pulling on the first end of the leader to cause the second swab to wipe the walls of the channel or lumen and absorb liquid in the channel or lumen.

### BRIEF DESCRIPTION OF THE DRAWINGS

FIG. 1 is a cross-sectional elevation view of a drying device in accordance with one embodiment of the invention.

FIG. 2 is a front and side perspective view of a drying device in accordance with the invention

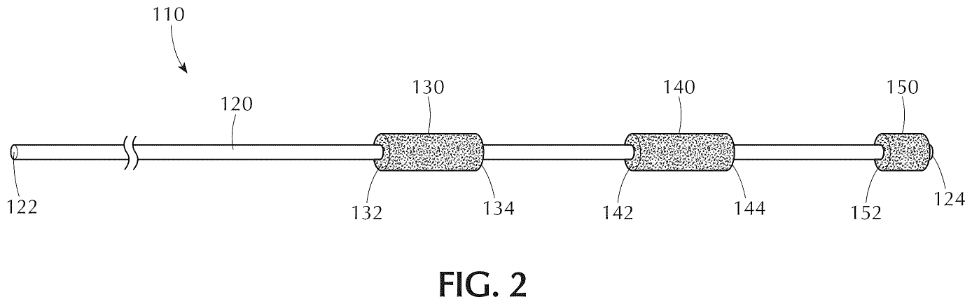

FIG. 3 is a perspective view of the use of a drying device in accordance with the invention to dry the channel or lumen of a flexible endoscope.

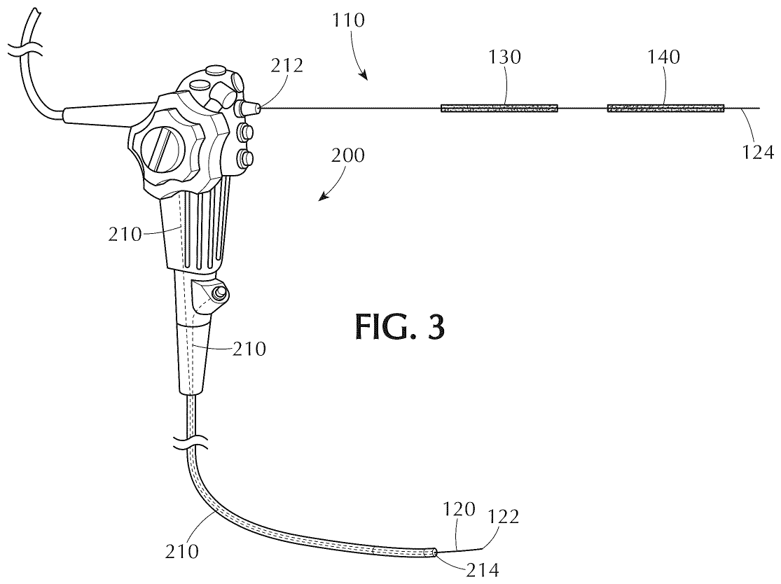

### DETAILED DESCRIPTION OF THE INVENTION

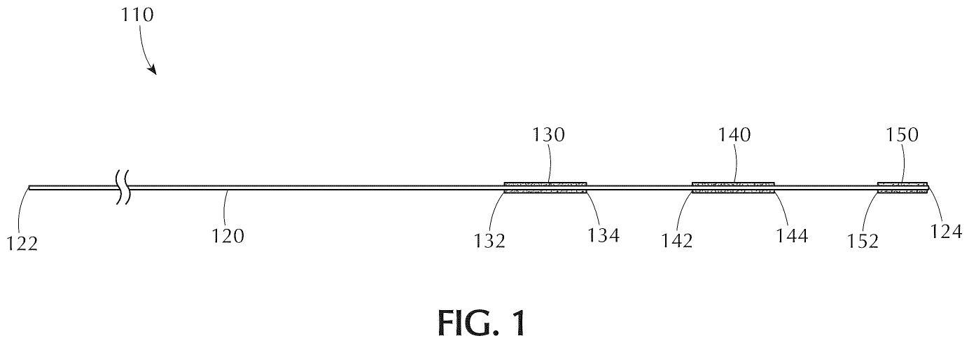

Referring to FIG. 1, a drying device 110 is shown. Drying device 110 is adapted for drying a channel or lumen 210 of a medical instrument, most particularly, surgical instruments such as an endoscope 200. As used herein, “endoscope” refers to the wide variety of flexible endoscopes and rigid endoscopes called by a wide variety of names and used for many different procedures, for example, laparoscopes, cystoscopes, esphagoscopes, sigmoidoscopes, duodenoscopes, gastroscopes, otoscopes, etc., as well as borescopes and other endoscope products used for non-medical procedures.

Drying device 110 has a flexible leader 120 having a first end 122 and a second end 124. The leader 120 is made of material with a reasonable rigidity or stiffness sufficient to pass it through the length of channel/lumen such as a 2 meter channel of 3.0 mm diameter. The desired stiffness may be achieved by utilizing a stiff polymer to form leader 120, e.g. PVC, or nylon, and by forming a monofilament thread or hollow tube. In some embodiments, a support wire (e.g., a conventional soft steel or iron wire) may be inserted into a hollow tube of leader, or a braided wire and polymer thread can be used to form leader 120. When wire is used, the leader 120 is preferably coated with a polymer layer or jacket (e.g., heat shrink tubing), preferably made of nylon. In preferred embodiments, leader 120 is a hollow tube made of polyvinyl chloride (PVC).

Leader 120 is preferably cylindrical in shape, having a generally consistent diameter. The diameter of the leader 120 is smaller than the diameter of the channel or lumen 210 of a medical instrument such as endoscope 200. Leader 120 thus is sized to be passed through the inside of the channel or lumen 120 without damaging the channel walls. Leader 120 has a diameter of from about 0.5 mm to about 4.0 mm, preferably about 1.5 mm to about 2.7 mm. In one preferred embodiment, leader 120 has a diameter of 1.5 mm.

Leader 120 is long enough to be passed all the way through the channel or lumen 210 of endoscope 200 so that a user can pass the first end 122 all of the way through the inside of the channel/lumen 210 so that the first end 122 extends out of the channel/lumen 210 end 214 which is opposite the receiving end 212 of the channel/lumen 210, while the second end 124 remains outside the receiving end of the channel/lumen 210. In embodiments for use with flexible endoscopes, leader 120 will have a length greater than 2 meters, preferably 2.2 to 2.6 meters, most preferably 2.4 meters. In embodiments for use with rigid endoscopes, the leader 120 will have a shorter length, preferably 35 cm to 150 cm, more preferably 40 to 80 cm. most preferably 50 cm. In most preferred embodiments, the flexible leader 120 has a diameter of 1.5 mm and a length of 2400 mm.

A first swab 130 formed of a hydrophobic non-absorbent resilient foam is affixed to the flexible leader 120. The position of first swab 130 on leader 120 is located so that the distance from the flexible leader first end 122 to the first swab 130 is greater than the length of the channel or lumen 210, but the first swab 120 is located away from the leader second end 124. First swab 130 is preferably formed of a hydrophobic polyurethane foam. For example, first swab 130 may be made from an open cell, flexible ether-based urethane foam; or an open cell, flexible polyester based polyurethane foam. Alternatively, a hydrophobic cross link polyethylene foam could be used.

A second swab 140 formed of a hydrophilic resilient foam is affixed to the flexible leader 120 between the first swab 130 and the second end 124. Second swab 140 is preferably formed of a hydrophilic polyurethane foam.

Optionally, a third swab 150 formed of a hydrophilic foam is affixed to the flexible leader 120 between the second swab 140 and the second end 124. If a desired, a fourth and/or fifth swab formed of a hydrophilic foam could be incorporated in the drying device 110 between the third swab 150 and the second end 124.

First swab 130 and second swab 140 (and any additional swabs) have a generally cylindrical shape. Specifically, the swabs 130, 140 have a hollow cylindrical shape, e.g. a tubular shape. Swabs 130 and 140 preferably have generally tapered first ends 132, 142 which are at the ends of the swabs 130, 140 which are closest to the leader first end 122, so that the tapered first ends 132, 142 are generally conical. Any additional swabs such as swab 150 may be cylindrical or may have a tapered first end 152.

The swabs 130, 140 (and any additional swabs) are affixed to leader 120. The swabs may be affixed to leader 120 by molding the swabs onto leader 120. In other embodiments, swabs may be molded as individual pieces each with a central bore, then slipped onto leader 120 with the leader fitted 120 into the central bore and positioned where desired and retained in position with an adhesive applied to the leader 120 at the desired location.

Channels in endoscopes typically have a diameter in the range of about 2.0 mm to about 4.5 mm. In some embodiments, first swab 130 and second swab 140 have a diameter of 4 millimeters or more, preferably 4.1 millimeters, for use with a 3.0 mm channel/lumen, but optionally larger (5.5 mm) for a larger 4.5 mm diameter lumen, or smaller (3.1 mm) for use with a smaller 2.0 mm channel/lumen.

In preferred embodiments, first swab 130 and second swab 140 have a length of 20 mm or more, preferably 40 mm.

In preferred embodiments, the distance from the flexible leader first end 122 to the first swab 130 is equal to the length of the channel or lumen, plus at least 20 mm, preferably 40 mm or more. In one embodiment, the distance from the flexible leader first end 122 to the first swab 130 is at least 1200 mm, preferably 1200-1400 mm.

The distance from the second end 134 of first swab 130 to the first end 142 of second swab 140 is desirably 40 mm to 80 mm. Similarly, the distance from the second end 144 of second swab 140 to the first end 152 of third swab 150 is also desirably 40 mm to 80 mm.

A method of use of the drying device 110 includes the steps of passing the first end 122 of the leader 120 through a first open end 212 of the channel or lumen 210 until the first end of the leader passes out of the channel or lumen 210 from a second end 214 of the channel or lumen; pulling on the first end 122 of the leader 120 to cause the first swab 130 to wipe the walls of the channel or lumen 210 and push liquid in the channel or lumen 210 out of the second end of the channel or lumen 210; and pulling on the first end 122 of the leader 120 to cause the second swab 140 to wipe the walls of the channel or lumen 210 and absorb liquid in the channel or lumen 210. If additional swabs are provided on the drying device 110, further pulling on the first end 122 of the leader 120 will cause the additional swabs (such as swab 150) to wipe the walls of the channel or lumen 210 and any remaining liquid in the channel or lumen 210.

A channel or lumen 210 of an endoscope 200 having a consistent diameter along its length will be most effectively dried by the drying device 110 of the present invention. Typically a suction or biopsy or combined suction/biopsy channel/lumen will have a consistent 3 millimeter diameter and will be the most suitable channels/lumens to be dried with the drying device. However, the drying device 110 of the present invention can be adapted for use in a channel/lumen having a varying diameter by providing a kit containing drying devices 110 of varying diameters which correlate to the diameters of the channel/lumen having a varying diameter, which drying devices can be used sequentially to obtain either partial or complete drying of the channel/lumen. Thus the present invention has a potential application in drying an air/water channel of an endoscope.

Use of a drying device 110 according to the present invention can be used to dry channels in an instrument such as an endoscope within minutes, which is substantially quicker than using forced air drying for 20-30 minutes or more. Further, the use of a drying device 110 allows the available forced air to be directed to other elements such as smaller channels in the endoscope, thus speeding up the drying process for the smaller channels as well as the channels dried with the drying device 110.

It should also be understood that the foregoing is illustrative and not limiting and that obvious modifications may be made by those skilled in the art without departing from the spirit of the invention. Accordingly, reference should be made primarily to the accompanying claims, rather than the foregoing specification, to determine the scope of the invention.

What is claimed is:

1. A channel or lumen drying device for a channel or lumen having a length, comprising:
a flexible leader having a first end and a second end;
a first swab, formed of a hydrophobic non-absorbent foam, affixed to the flexible leader at a position whereby a distance from the flexible leader first end to the first swab is greater than the length of the channel or lumen, and whereby the first swab position is away from the flexible leader second end;
a second swab, formed of a hydrophilic foam, affixed to the flexible leader between the first swab and the second end.

2. The channel or lumen drying device of claim 1, further comprising:
a third swab, formed of a hydrophilic foam, affixed to the flexible leader between the second swab and the second end.

3. The channel or lumen drying device of claim 1,
the first swab having a generally cylindrical shape; and
the second swab having a generally cylindrical shape.

4. The channel or lumen drying device of claim 1,
the first swab having a generally tapered first end which is closest to the flexible leader first end;
the second swab having a generally tapered first end which is closest to the flexible leader first end.

5. The channel or lumen drying device of claim 1, wherein:
the flexible leader is a cylindrical shape; and
the first swab having a hollow cylindrical shape wherein the first swab is mounted to the flexible leader.

6. The channel or lumen drying device of claim 5, the second swab having a hollow cylindrical shape wherein the second swab is mounted to the flexible leader.

7. The channel or lumen drying device of claim 5, wherein the flexible leader has a diameter of 1.5 millimeters and a length of 2000 millimeters or more.

8. The channel or lumen drying device of claim 7, wherein the first swab has a diameter of 4 millimeters or more and a length of 20 millimeters or more.

9. The channel or lumen drying device of claim 7, wherein second swab has a diameter of 4 millimeters or more and a length of 20 millimeters or more.

10. The channel or lumen drying device of claim 1, wherein the distance from the flexible leader first end to the first swab is 20 millimeters or more longer than the channel or lumen length.

11. A method of drying a channel or lumen having a first open end and a second open end and walls therebetween, using a channel or lumen drying device having a flexible leader having a first end and a second end, a first swab affixed to the flexible leader and located away from the second end, the first swab being a hydrophobic non-absorbent foam, and a second swab affixed to the flexible leader between the first swab and the second end, the second swab being a hydrophilic foam, comprising the steps of:
passing the first end of the leader through the first open end of the channel or lumen until the first end of the leader passes out of the channel or lumen from the second end of the channel or lumen;
pulling on the first end of the leader to cause the first swab to wipe the walls of the channel or lumen and push liquid in the channel or lumen out of the second end of the channel or lumen; and
pulling on the leader to cause the second swab to wipe the walls of the channel or lumen and absorb liquid in the channel or lumen.

12. The method of drying a channel or lumen of claim 11, wherein the channel or lumen drying device includes a third swab affixed to the flexible leader and between the second swab and the second end, the third swab being a hydrophilic foam, and the method further comprises:
pulling on the leader to cause the third swab to wipe the walls of the channel or lumen and absorb liquid in the channel or lumen.

13. The method of drying a channel or lumen of claim 11, wherein: the flexible leader is a generally solid or hollow cylindrical shape, the first swab having a hollow cylindrical shape wherein first swab is mounted to the flexible leader by receiving the flexible leader in the hollow of its hollow cylindrical shape, and the second swab having a hollow cylindrical shape wherein second swab is mounted to the flexible leader by receiving the flexible leader in the hollow of its hollow cylindrical shape.

14. The method of drying a channel or lumen of claim 13, the first swab having a generally tapered first end which is closest to the flexible leader first end; and the second swab having a generally tapered first end which is closest to the flexible leader first end.

15. The method of drying a channel or lumen of claim 11, wherein the flexible leader has a diameter of 1.5 millimeters and a length of 2000 millimeters or more.

16. The method of drying a channel or lumen of claim 11, wherein the first and second swabs have a diameter of 4 millimeters or more and a length of 20 millimeters or more.

17. The method of drying a channel or lumen of claim 11, wherein the distance from the flexible leader first end to the first swab is 20 millimeters or more longer than the channel or lumen length.
